(19) United States
(12) Patent Application Publication
(10) Pub. No.: US 2018/0279885 A1
(43) Pub. Date: Oct. 4, 2018

(54) DEVICE, SYSTEM AND METHOD FOR OBTAINING VITAL SIGN INFORMATION OF A SUBJECT

(71) Applicant: KONINKLIJKE PHILIPS N.V, EINDHOVEN (NL)

(72) Inventor: Murtaza BULUT, EINDHOVEN (NL)

(21) Appl. No.: 15/761,821

(22) PCT Filed: Oct. 7, 2016
(86) PCT No.: PCT/EP2016/074056

§ 371 Date: Mar. 21, 2018

(30) Foreign Application Priority Data
Oct. 8, 2015 (EP) 15188888.0

(51) Int. Cl. A61B 5/0205; A61B 5/00

(52) U.S. Cl. CPC A61B 5/0205; A61B 5/02427; A61B 5/7221; A61B 5/7275

(57) ABSTRACT

The present invention relates to device, system and method for obtaining vital sign information of a subject (14). To enable reliably obtaining vital sign information of a subject, in particular of the subject's heart rate, the proposed device comprises an input unit (21) for obtaining a set of image data (31) detected from a skin portion (24) of the subject (14) allowing the extraction of the subject's heart rate (51) and a set of detection data (31, 41) detected from a body portion of the subject (14) allowing the extraction of the subject's respiration rate (53) or representing the subject's respiration rate (53), a heart rate extraction unit (22) for extracting the subject's heart rate from said set of image data (31) by use of photoplethysmography, a prediction unit (23) for predicting changes of the subject's heart rate based on the subject's respiration rate extracted from or represented by said set of detection data, and a control unit (24) for controlling one or more settings of said heart rate extraction unit (22) based on the predicted changes of the subject's heart rate.

### FIELD OF THE INVENTION

The present invention relates to a device, system and method for obtaining vital sign information, in particular the heart rate, of a subject, such as person or animal.

### BACKGROUND OF THE INVENTION

Vital signs of a person, for example the heart rate (HR) or respiratory information (respiratory parameters) such as the respiratory rate (RR), can serve as a powerful predictor of serious medical events. For this reason the respiratory rate and/or the heart rate are often monitored online in intensive care units or in daily spot checks in the general ward of a hospital. Besides the heart rate, the respiratory rate is one of the most important vital signs. Both, the HR and the RR are still difficult to measure without having direct body contact. In present intensive care units, thorax impedance plethysmography or the respiratory inductive plethysmography are still the methods of choice for measuring the RR, wherein typically two breathing bands are used in order to distinguish thorax and abdominal breathing motion of a person. The HR is typically measured by use of electrodes, fixed at the chest of the subject, wherein the electrodes are connected to remote devices through cables. However, these obtrusive methods are uncomfortable and unpleasant for the patient being observed.

Moreover, unobtrusive respiratory rate measurements can be accomplished optically by use of a stationary video camera. A video camera captures the breathing movements of a patient's chest in a stream of images. The breathing movements lead to a temporal modulation of certain image features, wherein the frequency of the modulation corresponds to the respiratory rate of the patient monitored. Examples of such image features are the average amplitude in a spatial region of interest located around the patient's chest, or the location of the maximum of the spatial cross correlation of the region of interest in subsequent images.

Further, one or more video cameras are used for unobtrusively monitoring the HR, the RR or other vital signs of a subject by use of remote photoplethysmographic imaging. Remote photoplethysmographic imaging is, for instance, described in Wim Verkruysse, Lars O. Svaasand, and J. Stuart Nelson, “Remote plethysmographic imaging using ambient light”, Optics Express, Vol. 16, No. 26, December 2008. It is based on the principle that temporal variations in blood volume in the skin lead to variations in light absorptions by the skin. Such variations can be registered by a video camera that takes images of a skin area, e.g. the face, while the pixel average over a selected region (typically part of the cheek in this system) is calculated. By looking at periodic variations of this average signal, the heart rate and respiratory rate can be extracted. There are meanwhile a number of further publications and patent applications that describe details of devices and methods for obtaining vital signs of a patient by use of remote PPG.

Thus, the pulsation of arterial blood causes changes in light absorption. Those changes observed with a photodetector (or an array of photodetectors) form a PPG (photoplethysmography) signal (also called, among other, a pleth wave). Pulsation of the blood is caused by the beating heart, i.e. peaks in the PPG signal correspond to the individual beats of the heart. Therefore, a PPG signal is a heart rate signal in itself. The normalized amplitude of this signal is different for different wavelengths, and for some wavelengths it is also a function of blood oxygenation or other substances found in blood or tissue.

Moreover, unobtrusive non-camera based systems for obtaining vital sign information are also known. These systems are based on a surface structure comprising sensor units, which are in unobtrusive contact with the subject for obtaining vital sign information of the subject. Such systems are typically embodied in mattresses or textile structures, being in close proximity to the subject. The sensor units typically comprise pressure sensors for measuring pressure or weight distribution or time-dependent changes thereof and/or inductive sensors for measuring vital sign information, in particular ECG signals related to the heart rate or signals related to the respiration rate.

US 2014/0275832 A1 discloses a device for obtaining vital sign information of a subject. It comprises a first detection unit for acquiring first set of detection data allowing the extraction of a first vital sign information signal related to a first vital sign of the subject, a second detection unit for acquiring a second set of detection data allowing the extraction of a second vital sign information signal related to a second vital sign of the subject, an analysis unit for extracting the first vital sign information signal from the first set of detection data and for extracting the second vital sign information signal from the second set of detection data, a processing unit for combining the first vital sign information signal and the second vital sign information signal to obtain a combined vital sign information signal, and an extracting unit for extracting at least one of the first and second vital signs of the subject from the combined vital sign information signal.

One of the key challenges in such devices and method is to enable the determination of vital signs, in particular of the heart rate, reliably and accurately over the whole range of the heart rate spectrum, which is approximately from 30 to 240 bpm. In general this is a common problem of applications that try to monitor heart rate.

### SUMMARY OF THE INVENTION

It is an object of the invention to provide a device and a method as well as a system that enable reliably obtaining vital sign information of a subject, in particular of the subject's heart rate.

In a first aspect of the present invention a device for obtaining vital sign information of a subject is presented, said device comprising

an input unit for obtaining a set of image data detected from a skin portion of the subject allowing the extraction of the subject's heart rate and a set of detection data detected from a body portion of the subject allowing the extraction of the subject's respiration rate or representing the subject's respiration rate,

a heart rate extraction unit for extracting the subject's heart rate from said set of image data by use of photoplethysmography,

a prediction unit for predicting changes of the subject's heart rate based on the subject's respiration rate extracted from or represented by said set of detection data, and

a control unit for controlling one or more settings of said heart rate extraction unit based on the predicted changes of the subject's heart rate.

In a further aspect of the present invention a corresponding method for obtaining vital sign information of a subject is presented.

In a further aspect of the present invention a system for obtaining vital sign information of a subject is presented, said system comprising

an imaging unit for detecting a set of image data from a skin portion of the subject, said image data allowing the extraction of the subject's heart rate,

a detection unit for detecting a set of detection data from a body portion of the subject, said detection data allowing the extraction of the subject's respiration rate or representing the subject's respiration rate, and

a device as disclosed herein for obtaining vital sign information of the subject based on the detected set of image data and the detected set of detection data.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed method has similar and/or identical preferred embodiments as the claimed device, as defined in the dependent claims and as disclosed herein.

To be able to cope with the wide range of possible frequencies known devices, systems and methods are generally equipped with different modules that can perform well in average, but they compromise beat-to-beat accuracy, or increase complexity, or decrease processing speed. In other words, they do make use of the expected heart rate changes to dynamically adapt the system parameters (herein meaning one or more parameters of the heart rate extraction unit, i.e. one or more parameters that are relevant for the heart rate extraction). By having some prior information about the expected heart rate changes, the possible HR band can be limited, and therefore the system parameters can be selected so that the beat to beat accuracy is higher.

Hence, the present invention is based on the idea to obtain information on expected changes of the heart rate from respiration rate changes and to select the system parameters accordingly. In particular, one or more settings of the heart rate extraction unit are controlled based on the predicted changes of the subject's heart rate, which, in turn, is derived from at least the subject's respiration rate. This is possible since changes in respiration rate and heart rate are positively correlated. Heart and respiratory rates usually move together because they both respond to oxygen demand. Events that cause increase in respiration rate also cause changes in heart rate.

In an embodiment said control unit is configured to control one or more settings of said heart rate extraction unit based on the predicted changes of the subject's heart rate and the actual changes of the subject's heart rate. Hence, in addition to the predicted heart rate the actual heart rate is used, for instance to check if the actual heart rate behaves as predicted or not and to control the settings accordingly.

In another embodiment said prediction unit is configured to determine a reliability indicator (also called confidence measure) indicating the reliability of the extracted heart rate based on the predicted changes of the subject's heart rate and the actual changes of the subject's heart rate. For instance, the reliability is considered to be higher if the actual heart rate corresponds to the predicted heart rate.

The heart rate extraction unit may be configured to derive a photoplethysmography, PPG, signal from the set of image data, and the control unit may be configured to control the window length of a window of the PPG signal and the window length of the short-time window used in an analysis of the PPG signal. This window should be selected in manner that it is long enough to include at least one full period of the heart rate cycle and should be short enough so that more signals are available for processing. The window may be a short-time analysis window, which is a window that is used in an algorithm, such as FFT, to extract the heart rate signal. The window may also be a time window, which is a window, for which data are acquired, e.g. image data, a PPG signal or another time signal. This time signal may be processed by an algorithm operating on in time domain, such as wavelets, or by a different algorithm operating in frequency domain such as FFT.

In another embodiment said set of detection data further allows the extraction of or includes the subject's inhalation time and exhalation time, wherein said control unit is configured to control one or more settings of said heart rate extraction unit based on the predicted changes of the subject's heart rate and the subject's inhalation time and exhalation time. Generally, during the inhalation the heart rate increases and during exhalation the heart rate decreases. The settings of the heart rate extraction unit can be controlled accordingly, if the inhalation time and the extraction time are known.

Hence, said control unit may be configured to control one or more settings of said heart rate extraction unit such that different settings are applied during the subject's inhalation time compared to the subject's exhalation time. Further, said prediction unit may be configured to determine a reliability indicator indicating the reliability of the extracted heart rate based on the predicted changes of the subject's heart rate and the actual changes of the subject's heart rate during the subject's inhalation and during the subject's exhalation.

A determined reliability indicator may also be used in the control of the settings to further improve the efficiency of the control. For instance, the control unit may be configured to change one or more settings of said heart rate extraction unit if the current reliability indicator value is below a predetermined threshold or is lower than the preceding reliability indicator value.

In preferred embodiments, it is proposed to observe changes in factors that can influence heart rate, in particular environmental parameters, such as altitude and temperature of the subject's location, and subject-related parameters, such as sweating, body position, activity (such as walking, running, sleeping, etc.), optionally other vital signs like body temperature, SpO2, blood pressure, etc. These changes are used additionally in the prediction of the heart rate changes and thus in the control of the settings of the heart rate extraction unit. By introducing more informed parameters the heart rate detection accuracy can be increased and an improved confidence metric can be generated for the produced results. Accordingly, the control unit is configured to control one or more settings of said heart rate extraction unit based on the predicted changes of the subject's heart rate and one or more additional indicators including one or more of a sweating indicator indicating sweating of the subject, an activity indicator indicating activity of the subject, a posture indicator indicating body posture or position of the subject, an altitude indicator indicating altitude of the subject's location, and a temperature indicator indicating temperature at the subject's location.

The device may further comprise a health state determining unit for determining the health state of the subject based on the current heart rate, the current respiration rate and the current one or more additional indicators compared to earlier heart rates, respiration rates and one or more additional indicators. Thus, the available information may be favorably used for obtaining additional information, i.e. the subject's health state.

In another embodiment said control unit is configured to control one or more settings of said heart rate extraction unit based on the predicted changes of the subject's heart rate and based on the identity of the subject obtained as input via the input unit or recognized by a recognition unit. Hence, the control can be personalized to improve its accuracy and efficiency.

Still further, in an embodiment said set of detection data corresponds to said set of image data, wherein said device further comprises a respiration rate extraction unit for extracting the subject's respiration rate from said set of image data, e.g. by detecting motion of a subject's body portion, e.g. of the subject's chest or belly, showing motion that is caused by respiration. The image data is preferably acquired by an imaging unit, which may thus be further adapted to acquire a separate set of image data, being detected from a body portion of the subject allowing the extraction of a motion signal related to the respiratory rate information of the subject. By way of example, a body portion is typically the chest of the person or the nose or even other areas of the body of the subject, where respiratory motion can be detected.

In other embodiments one or more separate sensors may be provided, e.g. unobtrusive sensors, such as capacitive sensor and/or pressure sensors, or conventional respiration sensors, to obtain detection data from the subject, from which the respiration rate can be extracted.

The present invention can be employed in devices and systems using remote PPG, e.g. by use of a remote vital signs camera, but also in other devices and systems that monitor heart rate.

### BRIEF DESCRIPTION OF THE DRAWINGS

These and other aspects of the invention will be apparent from and elucidated with reference to the embodiments described hereinafter. In the following drawings

FIG. 1 shows a first, schematic embodiment of a system for obtaining vital sign information of a subject according to the present invention including a first, schematic embodiment of a device according to the present invention,

FIG. 2 shows a second embodiment of a system for obtaining vital sign information of a subject according to the present invention,

FIG. 3 shows a second embodiment of a device for obtaining vital sign information of a subject according to the present invention,

FIG. 4 shows a third embodiment of a system for obtaining vital sign information of a subject according to the present invention,

FIG. 5 shows a third embodiment of a device for obtaining vital sign information of a subject according to the present invention,

FIG. 6 shows a fourth embodiment of a system for obtaining vital sign information of a subject according to the present invention,

FIG. 7 shows a fourth embodiment of a device for obtaining vital sign information of a subject according to the present invention,

FIG. 8 shows a fifth embodiment of a device for obtaining vital sign information of a subject according to the present invention, and

FIG. 9 shows a flow chart of a method for obtaining vital sign information of a subject according to the present invention.

### DETAILED DESCRIPTION OF THE INVENTION

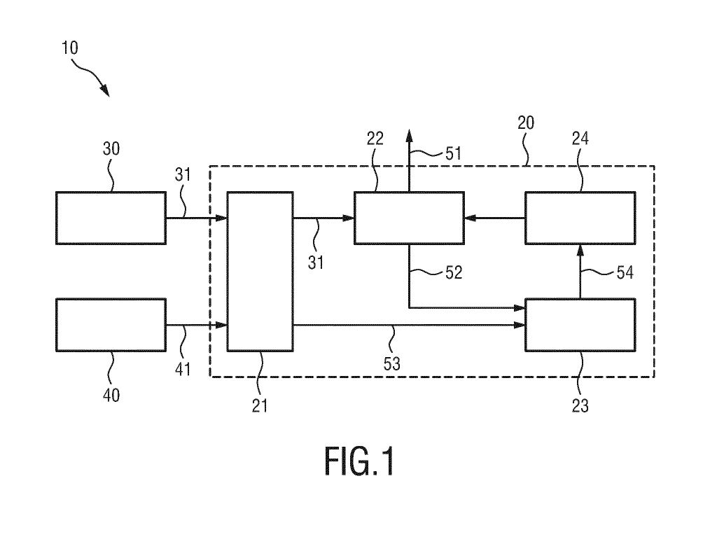

FIG. 1 shows a first, schematic embodiment of a system 10 for obtaining vital sign information of a subject according to the present invention including a first, schematic embodiment of a device 20 according to the present invention. Besides the device 20 the system 10 comprises an imaging unit 30 for detecting a set of image data 31 from a skin portion of the subject, said image data 31 allowing the extraction of the subject's heart rate. Further, the system 10 comprises a detection unit 40 for detecting a set of detection data 41 from a body portion of the subject, said detection data 41 allowing the extraction of the subject's respiration rate or representing the subject's respiration rate.

The device 20 comprises an input unit 21, e.g. a data interface, for obtaining the set of image data 31 and the set of detection data 41. A heart rate extraction unit 22 extracts the subject's heart rate 51 from said set of image data 31 by use of photoplethysmography, which is a commonly known principle as explained above. A prediction unit 23 predicts changes 52 of the subject's heart rate based on the subject's respiration rate 53 (which is derived from the detection data 41 or directly corresponds to the detection data 41). A control unit 24 controls, e.g. via a control signal 54, one or more settings of said heart rate extraction unit 22 based on the predicted changes 52 of the subject's heart rate.

The various units of the device 20 may be comprised in one or multiple digital or analog processors depending on how and where the invention is applied. The different units may completely or partly be implemented in software and carried out on a personal computer connected to one or more detectors. Some or all of the required functionality may also be implemented in hardware, e.g. in an application specific integrated circuit (ASIC) or in a field programmable gate array (FPGA).

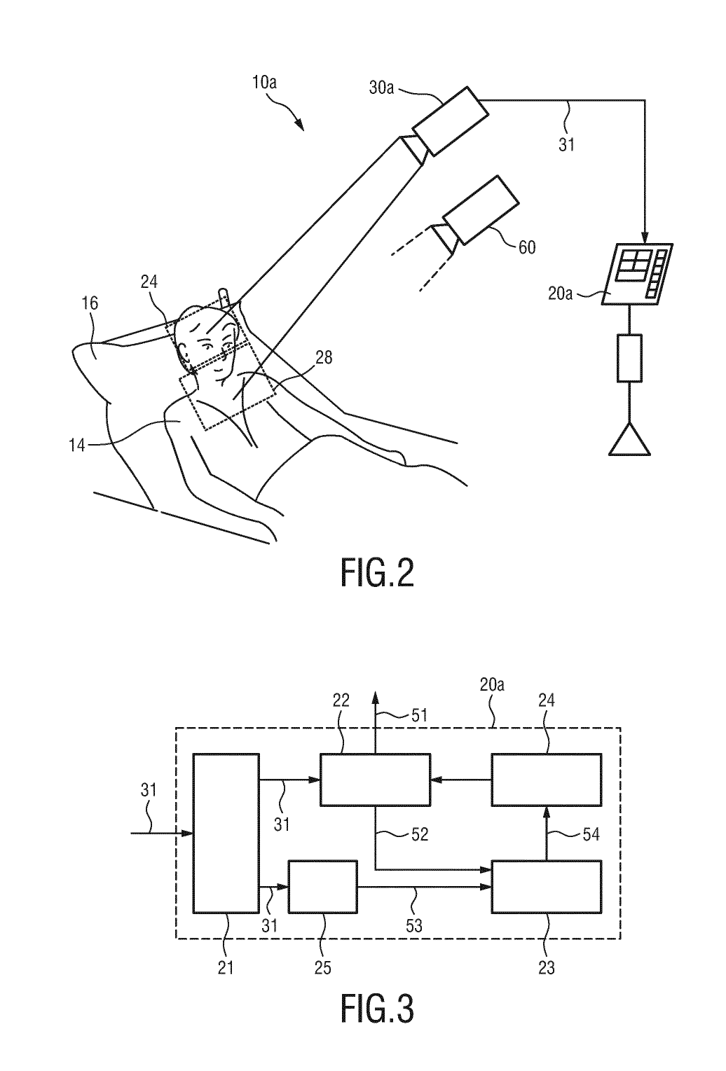

FIG. 2 shows a second embodiment of a system 10a according to the present invention including a second embodiment of a device 20a according to the present invention as schematically depicted in FIG. 3. The subject 14, in this example a patient, lies in a bed 16, e.g. in a hospital or other healthcare facility, but may also be a neonate or premature infant, e.g. lying in an incubator, or person at home or in a different environment.

Besides the device 20a, the system 10a further comprises an imaging unit 30a for detecting a set of image data 31. The image data 31 hereby include image data from a skin portion 17 of the subject 14, e.g. from the face, allowing the extraction of the subject's heart rate. Further, the image data 31 include image data, representing the detection data 41, from a body portion 18 of the subject 14, e.g. from the chest, allowing the extraction of the subject's respiration rate (e.g. via the detection of periodic motion of the chest wall, as is commonly known, e.g. from US 2014/0275832 A1. For this purpose the device 20a further comprises a respiration rate extraction unit 25 to determine the subject's respiration rate 53 from the image data 31. Hence, in this embodiment the imaging unit 30a commonly represents the imaging unit 30 and the detection unit 40 of the system 10.

In this embodiment, the skin portion 17 may be the forehead of the subject 14 and the body portion 18 may be the chest of the subject 14. It is to be understood that in other embodiments, the skin portion 17 can also be the arm or other detectable skin areas of the subject and the body portion can also include the mouth and/or the nose of the subject 14.

The imaging units 30, 30a preferably comprise a camera (also referred to as camera-based or remote PPG sensor) including a suitable photosensor for (remotely and unobtrusively) capturing image frames of the subject 14, in particular for acquiring a sequence of image frames of the subject 14 over time, from which photoplethysmography signals (PPG signals) can be derived. The image frames captured by the camera may particularly correspond to a video sequence captured by means of an analog or digital photosensor, e.g. in a (digital) camera. Such a camera usually includes a photosensor, such as a CMOS or CCD sensor, which may also operate in a specific spectral range (visible, IR) or provide information for different spectral ranges. The camera may provide an analog or digital signal. The image frames include a plurality of image pixels having associated pixel values. Particularly, the image frames include pixels representing light intensity values captured with different photosensitive elements of a photosensor. These photosensitive elements may be sensitive in a specific spectral range (i.e. representing a specific color). The image frames include at least some image pixels being representative of a skin portion of the subject. Thereby, an image pixel may correspond to one photosensitive element of a photodetector and its (analog or digital) output or may be determined based on a combination (e.g. through binning) of a plurality of the photosensitive elements.

In this setup, the imaging unit 30a is installed at a remote distance, for example at a ceiling or a wall of a room in which the bed 16 is located. An additional light source 60 can be present to illuminate the scenery and to ensure sufficient image contrast. In one embodiment, the imaging unit 30a can be an infrared camera and the light source 60 can be an infrared light source. It is to be understood, that in further embodiments the imaging unit 30a can be adapted to detect light in the visible or infrared spectral range and the light source 60 can be adapted to emit light in the infrared and/or visible spectral range. In this embodiment, the subject 14 and the imaging unit 30a are located opposite to one another. It is to be understood that the imaging unit 30a and/or the light source 60 can in principle be arbitrarily oriented with respect to the subject 14.

The present invention exploits that heart rate calculations can benefit from respiration calculations, e.g. by use of single vital signs camera setup as illustrated in FIG. 2. One of the ideas is to update the heart rate calculation settings based on the observed changes in the respiration rate, and in general, in the state of the measured subject. This is possible since heart rate and respiration rate are linked.

Human body, and essentially all living organisms, are one complete system where everything is linked. The present invention relates to the interaction between cardiovascular and respiratory systems, and particularly to heart rate and respiration rate.

Respiratory sinus arrhythmia (RSA) is a naturally occurring variation in heart rate that occurs during a breathing cycle. The breathing cycle consists of inhalation and exhalation periods. During this cycle, inhalation temporarily suppresses vagal activity, causing an immediate increase in heart rate. Exhalation then decreases heart rate and causes vagal activity to resume. In short, heart rate increases during the inhalation, and decreases during exhalation. The changes in the heart rate can be up to 20 bpm or more in some cases.

When muscles, even the heart, are working harder (i.e. during an activity), they are also burning more calories. The muscles need more oxygen than they normally use to burn these extra calories. To deliver more oxygen, the breathing rate increases to bring more oxygen into the lungs. Heart rate also increases so that more oxygen is delivered to the muscles. In short, changes in respiration rate and heart rate are positively correlated. Heart and respiratory rates usually move together because they both respond to oxygen demand. Events that cause increase in respiration rate also cause changes in heart rate.

Fit people are able to carry out physical activities more effectively than unfit people. Their pulse rate is likely to return to normal more quickly after exercise. In other words, the rate of change of heart and respiration rate in fit people is less in comparison to unfit people.

In addition to the interaction between heart rate and respiration rate, there is a need to also underline other environmental factors that influence the heart rate. In particular, at high altitudes, particularly when a person first arrives there, it is normal for heart rate and breathing rate to increase. Usually, once acclimated, the heart and respiratory rate should return to normal. Further, when a person stands up, heart rate rises and leg blood vessels tighten to maintain blood flow to your brain. Breathing is less affected. If you have heart failure, breathing and heart rate may rise upon lying down since more blood returns to the heart than it can handle and it backs up into the lungs. Still further, temperature affects a large amount of the processes that occur in the human body. Warmer temperatures cause the heart to beat faster and place considerable strain on the body. Simply put, when it is hot, the body must move more blood to the skin to cool it while also maintaining blood flow to the muscles. The only way to do both of these things is to increase overall blood flow, which means that the heart must beat faster. Depending on how fit one is and how hot it is, this might mean a heart rate that is 20 to 40 bpm higher than normal.

The present invention provides that the system parameters of the device 20, in particular of the heart rate extraction unit 22, are set such that the device 20 can perform reliably accurately over the whole range of heart beat spectrum, which is approximately from 30 to 170 bpm. This is achieved by using some prior information about the expected heart rate changes, so that the possible heart rate band can be limited, and therefore the system parameters can be selected so that the beat to beat accuracy is higher. To predict the expected changes in the heart rate respiration rate changes are monitored according to the present invention, in order to select the system parameters accordingly.

The camera-based respiration monitoring as used in the above described second embodiment of the system and method is generally realized by measuring the subtle breathing motion in the subject's chest (or belly) area. Thus, it critically depends on the detection of subtle breathing motion in the image data. The motion-based respiratory signal monitoring is not always reliable, due to the difficulty of breathing motion detection in certain cases. For example, a neonate in the NICU sometimes has shallow breathing, where it is challenging to detect the very subtle breathing motion. If the algorithm parameters are adjusted to be sensitive enough to the very subtle motion of shallow breathing, another problem will arise: the algorithm may not be able to differentiate the subtle breathing motion from the noises (illumination, camera, etc.). For instance, pointing to the wall, the algorithm can produce the respiration-like signal due to the noises.

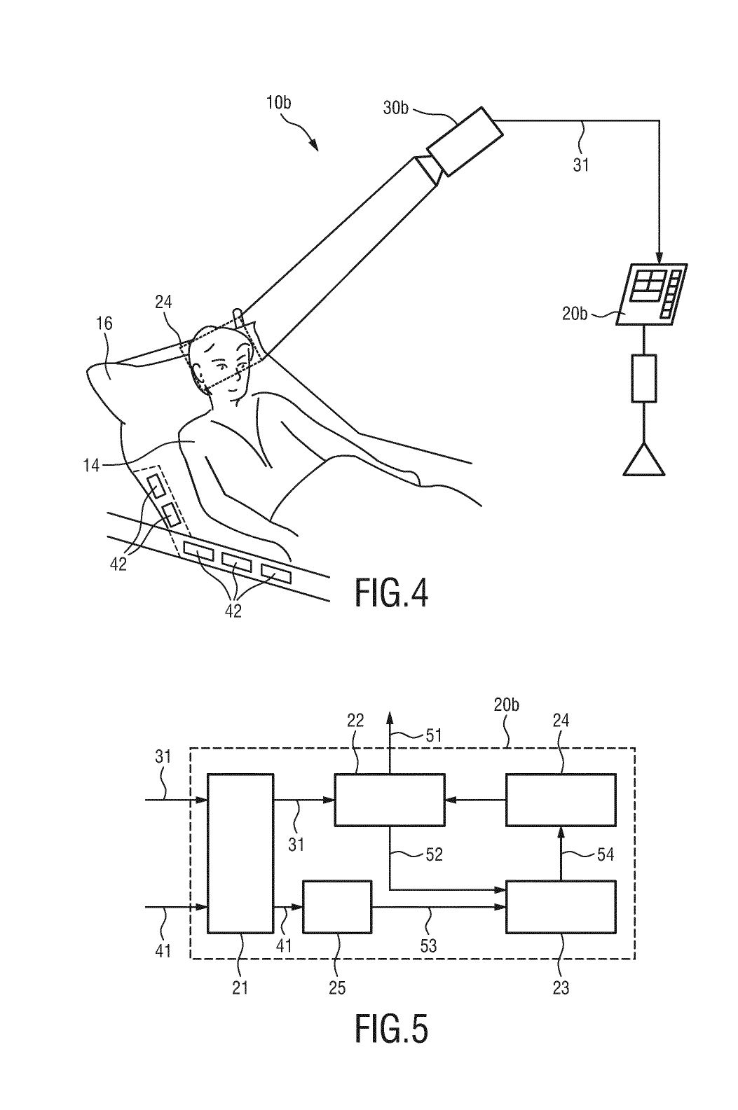

Hence, in a third embodiment of the system 10b and the device 20b illustrated in FIGS. 4 and 5 a separate detection unit 40b is provided for acquiring detection data 41 allowing the extraction of the subject's respiration rate or representing the subject's respiration rate in addition to the imaging unit 30b for acquiring image data 31 from a skin portion 17 of the subject 14, e.g. from the face, allowing the extraction of the subject's heart rate. The detection unit 40b may e.g. comprise a plurality of unobtrusive sensors 42 embedded into the bed, e.g. in the mattress. Such sensors 42 may e.g. include pressure sensors measuring a signal representative of body motion of the subject's body, e.g. of the chest wall, caused by the respiratory movements. It is to be understood that the sensors 42 can also be integrated into a textile structure of the bed, such as the blanket or the pillow, or into textiles worn by the subject 14. The respiration rate can thus be derived from the absolute pressure or pressure variations detected as sensor data by the pressure sensors 42 caused by the subject 14.

In a practical embodiment using (only) the respiration rate to adjust heart rate calculation parameters, at time T0 (beginning) device 20 operates as normal and measures the respiration rate (RR0) and heart rate (HR0) for the time window T0. At time T1, new respiration rate (RR1) and heart rate (HR1) parameters are calculated. It is proposed that at this stage, before HR1 is calculated, to do the following:

(i) Calculate diff_RR=RR1−RR0;
(ii) If diff_RR>0, the expectation is that HR1 should be higher than HR0;
(iii) Change the settings for the device, in particular of the heart rate extraction unit 22 to favor higher HR frequencies.
Alternatively, it can first be checked whether HR1>HR0, and if it is the case only then change the settings. In addition, a confidence measure (also called reliability indicator) based on calculation with two different settings can be also calculated.

Further, in an embodiment it is proposed to change the settings that relate to the decomposition of the trace signal into more nicely behaving components. In an implementation a signal decomposition algorithm is used to decompose the intensity signals from image data (e.g. video, also called traces). In the decomposition algorithm, one important parameter is the size of the short-time window. This window may be selected in a manner that it is long enough to include at least one full period of the heart rate cycle, and short enough so that more window segments are available for processing.

Hereby, the short-time window length settings can be changed as follows:

(i) if HR is expected to increase, then the short-time window size is decreased and
(ii) if the HR is expected to decrease, then the short-time window size is increased.
In addition or as an alternative to adjusting the short-time window based decomposition algorithms settings, the time window length parameter (of a window, for which data are acquired, e.g. image data, a PPG signal or another time signal) may be also changed. If lower heart rates are expected, the window length can be increased.

In an exemplary implementation, a time window size of 80 samples (4 seconds) and a short-time window size of 30 samples (1.5 seconds) may be used. It has been observed that these settings perform reliably over a broad range of frequencies. However, setting the short-time window size to 20 samples (1 second) improves the accuracy for higher HR frequencies (90 bpm and up), and increasing the short-time window size to 40 samples (2 seconds) and the time window size to 120 samples (6 seconds) improves the results for lower heart rate frequencies (60 bpm and lower).

In another embodiment not only the respiration rate, but also inhalation time and/or exhalation time are used to improve heart rate calculations. Hence, from the detection data, which, as mentioned above, may also be the image data, the subject's inhalation time and exhalation time are derived, e.g. by the respiration rate extraction unit 25 or another unit of the device, and the control unit 24 is configured to control one or more settings of said heart rate extraction unit 22 based on the predicted changes of the subject's heart rate and the subject's inhalation time and exhalation time. Generally, during the inhalation the HR increases and during exhalation the HR decreases. Having this physiological truth, the system parameters can be adapted to favor higher HR during inhalation and lower HR during exhalation. Alternatively, the system parameters can be kept unchanged, but HR calculations during these periods can be compared. Hence, one or more settings of said heart rate extraction unit 25 are controlled such that different settings are applied during the subject's inhalation time compared to the subject's exhalation time

If it is observed that HR during inhalation is larger than HR during exhalation, the confidence in the calculated HR (and the corresponding settings) is increased. Otherwise, if it is observed that HR during inhalation is lower that HR during exhalation, the calculated HR is discarded, or confidence in the results decreased, or system parameters are updated. Hence, the control unit 24 is configured to change one or more settings of said heart rate extraction unit 22 if the current reliability indicator value is below a predetermined threshold or is lower than the preceding reliability indicator value.

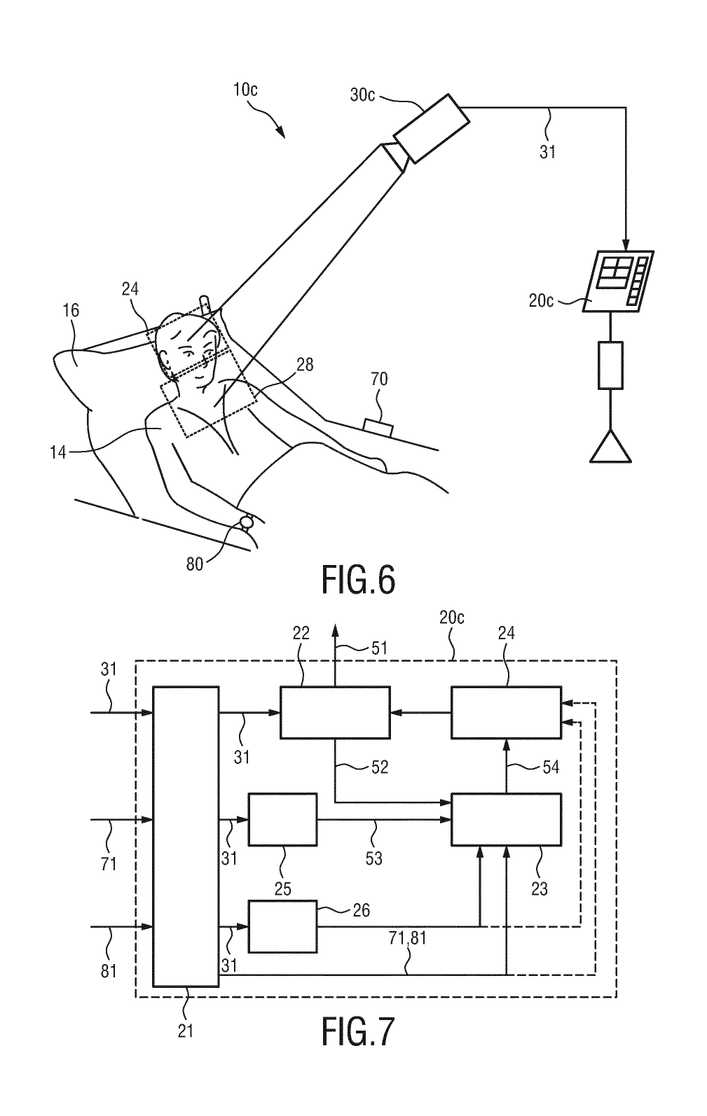

As explained above, in a preferred embodiment further parameters, in particular environmental parameters and/or subject-related parameters, that can influence heart rate, are additionally in the prediction of the heart rate changes and thus in the control of the settings of the heart rate extraction unit. Corresponding embodiments of a system 10c and a device 20c are shown in FIGS. 6 and 7. In this embodiment the system 10c comprises one or more additional sensor 70, 80 for acquiring corresponding sensor data 71, 81 (as explained in more detail below), which are then used in addition by the control unit 24, optionally after further processing if needed, to control the settings of the heart rate extraction unit 22. Hereby, additional sensor data 71, 81 (optionally after processing) are directly used by the control unit 24 or are used as additional input in the prediction of HR changes by the prediction unit 23.

In one embodiment, sweating of the subject 14 is additionally detected and used in the control. Sweating can e.g. be detected easily from the image data 31 of the camera 30a, from the PPG signal characteristics and/or from video analysis, for which purpose an additional image processing unit 26 may be provided. Alternatively, a dedicated sweat sensor may be used, e.g. incorporated in a wrist-worn device 80 providing additional sensor data 81. Such a sweat sensor may e.g. be a sensor that measures the electrodermal activity (EDA), which is the property of the human body that causes continuous variation in the electrical characteristics of the skin, in particular that measures a parameter like skin conductance, galvanic skin response (GSR), etc. indicating the amount of sweating of the subject. Sweating increases the heart rate. When sweating is detected the system parameters can be adjusted to favor higher heart rates.

In another embodiment, activity of the subject 14 is additionally used in the control. Activity can e.g. be detected from the image data 31 of the camera 30a by use of the image processing unit 26. If it is detected that the person has started a new activity that requires change in physical effort, the HR parameters can be changed to better calculate the expected HRs. For example, if it is observed that the person is first walking, and then start running, it is expected for HR to increase so that the system settings can be adjusted accordingly. When the person is running settings that favor higher HR values (e.g. a smaller short-time window length) are used.

In another embodiment, the body position or posture is additionally used in the control. Changes in body position or posture, e.g. sitting vs. standing vs. lying, can e.g. be detected from the image data 31 of the camera 30a by use of the image processing unit 26. The system parameters can be changed accordingly. The HR when sitting is lower than the HR when standing. The difference is around 10 beats per minute. Hence, if a person is sitting and HR has been measured as X with high confidence, it can be safely assumed that when he stands (after standing) that his HR will not be lower than this measured value X. However, this assumption does not hold when there is a big time difference between sitting and standing.

Here, the focus is particularly on the transition from sitting to standing and vice versa. For example, when the person goes from sitting to standing the length of the short-time window can be decreased momentarily to account for the HR increase prediction. If the person has been standing for a while, then the window size return to “average” settings, which corresponds to the best calculation of the average HR of the person.

In another embodiment, the altitude of the subject's location is additionally used in the control. The altitude can e.g. be detected by an altitude sensor 70, which may optionally also be included in the wrist-worn device 80, to provide altitude data 71. When altitude increases the HR and respiration rate increase. After the person adapts to new conditions, they return to normal. This adaptation time can be also taken as an additional parameter in the control of the settings of the HR extraction. In generally, use may be made of the historical data for the person as well, as an additional input for the control unit.

In another embodiment, the temperature (or temperature changes) of the subject's location is additionally used in the control. Before the body adapts to temperature changes, the body reacts to them. An increase in environment temperature is expected to result in increased heart rate. This information can be taken into account, and the HR calculation settings can be adjusted accordingly.

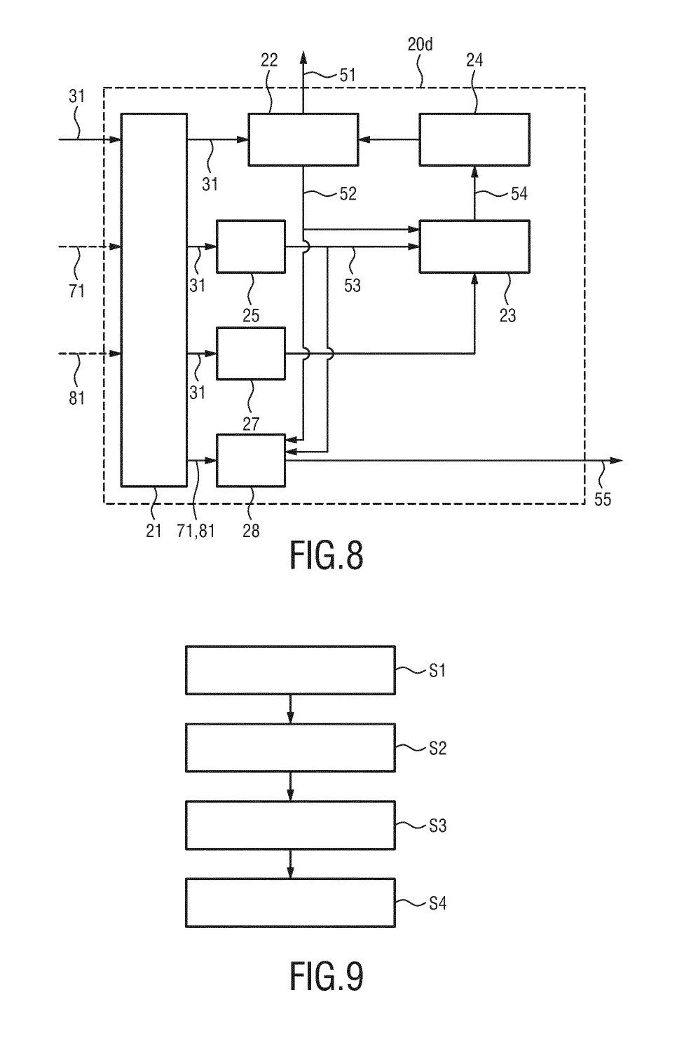

In still another embodiment, the control of the HR extraction may be personalized to the subject 14. Personalization may e.g. be achieved by receiving identity data of the subject 14 as input or by recognizing the subject 14 from the image data 31 by use of a recognition unit 27, e.g. a face recognition unit, as conventionally known. This is illustrated in a fifth embodiment of the device 20d illustrated in FIG. 8. The expected changes in the HR can thus be personalized, and the system parameters can be changed accordingly. For example, if it known (from prior data, e.g. stored in a storage unit in the device, in the system or e.g. in a hospital archive) that for person A, 10° C. increase in temperature results in 40 bpm increase in HR, versus person B for whom only 10 bpm increase happens for 10° C. increase in temperature, the system parameters can be changed differently for person A and person B. The same is true for the other factors that influence HR, such as respiration, activity, altitude, sweating, etc. Hence, it is advantageously exploited according to this embodiment to know the relative changes in the ratio between respiration and heart rate as well as other factors that affect the HR. Recognizing the person and updating the system parameters and optionally confidence metric calculations accordingly can significantly improve the accuracy of the HR extraction.

In still another embodiment the device 20d further comprises a health state determining unit 28 for determining the health state 55 of the subject 14 based on the current heart rate, the current respiration rate and the current one or more additional indicators compared to earlier heart rates, respiration rates and one or more additional indicators. Thus, the correlation between respiration and heart signal parameters can be used as a means to track the health state of the subject 14. If the calculated respiration parameters (respiration rate, inhalation time and/or exhalation time), heart signal parameters (heart rate), environment parameters (temperature and/or altitude), and activity parameters (sweating, body position and/or activity) are stored for a duration of time, the changes in their relation can be used to infer changes in the health state of the subject. For example, if it is observed that, compared to two months ago, running does not increase heart rate and respiration rate as much, it can be induced that the subject's physical condition has improved. In another example, if it is observed that increase in respiration rate caused larger increase in heart rate compared to the past, it can be induced that health state of the subject has changed negatively.

FIG. 9 shows a flow chart of a method for obtaining vital sign information of a subject according to the present invention. In a first step S1 a set of image data detected from a skin portion of the subject is obtained, said image data allowing the extraction of the subject's heart rate and a set of detection data detected from a body portion of the subject allowing the extraction of the subject's respiration rate or representing the subject's respiration rate. In a second step S2 the subject's heart rate is extracted from said set of image data by use of photoplethysmography. In a third step S3 changes of the subject's heart rate are predicted based on the subject's respiration rate. In a fourth step S4 one or more settings of said heart rate extraction unit are controlled based on the predicted changes of the subject's heart rate. The method can be adapted further in accordance with the embodiments explained above for the proposed device and system.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A device for obtaining vital sign information of a subject, said device comprising:
an input unit for obtaining a set of image data detected from a skin portion of the subject allowing the extraction of the subject's heart rate and a set of detection data detected from a body portion of the subject allowing the extraction of the subject's respiration rate or representing the subject's respiration rate,
a heart rate extraction unit for extracting the subject's heart rate from said set of image data by use of photoplethysmography,
a prediction unit for predicting changes of the subject's heart rate based on the subject's respiration rate extracted from or represented by said set of detection data, and
a control unit for controlling one or more settings of said heart rate extraction unit based on the predicted changes of the subject's heart rate.

2. The device as claimed in claim 1,
wherein said control unit is configured to control one or more settings of said heart rate extraction unit based on the predicted changes of the subject's heart rate and the actual changes of the subject's heart rate.

3. The device as claimed in claim 1,
wherein said prediction unit is configured to determine a reliability indicator indicating the reliability of the extracted heart rate based on the predicted changes of the subject's heart rate and the actual changes of the subject's heart rate.

4. The device as claimed in claim 1,
wherein said heart rate extraction unit is configured to derive a photoplethysmography, PPG, signal from the set of image data and wherein said control unit is configured to control the window length of a window of the PPG signal and the window length of the short-time window used in an analysis of the PPG signal.

5. The device as claimed in claim 1,
wherein said set of detection data further allows the extraction of or includes the subject's inhalation time and exhalation time, wherein said control unit is configured to control one or more settings of said heart rate extraction unit based on the predicted changes of the subject's heart rate and the subject's inhalation time and exhalation time.

6. The device as claimed in claim 5,
wherein said control unit is configured to control one or more settings of said heart rate extraction unit such that different settings are applied during the subject's inhalation time compared to the subject's exhalation time.

7. The device as claimed in claim 5,
wherein said prediction unit is configured to determine a reliability indicator indicating the reliability of the extracted heart rate based on the predicted changes of the subject's heart rate and the actual changes of the subject's heart rate during the subject's inhalation and during the subject's exhalation.

8. The device as claimed in claim 3,
wherein said control unit is configured to change one or more settings of said heart rate extraction unit if the current reliability indicator value is below a predetermined threshold or is lower than the preceding reliability indicator value.

9. The device as claimed in claim 1,
wherein said control unit is configured to control one or more settings of said heart rate extraction unit based on the predicted changes of the subject's heart rate and one or more additional indicators including one or more of a sweating indicator indicating sweating of the subject, an activity indicator indicating activity of the subject, a posture indicator indicating body posture or position of the subject, an altitude indicator indicating altitude of the subject's location, and a temperature indicator indicating temperature at the subject's location.

10. The device as claimed in claim 9,
further comprising a health state determining unit for determining the health state of the subject based on the current heart rate, the current respiration rate and the current one or more additional indicators compared to earlier heart rates, respiration rates and one or more additional indicators.

11. The device as claimed in claim 1,
wherein said control unit is configured to control one or more settings of said heart rate extraction unit based on the predicted changes of the subject's heart rate and based on the identity of the subject obtained as input via the input unit or recognized by a recognition unit.

12. The device as claimed in claim 1,
wherein said set of detection data corresponds to said set of image data, wherein said device further comprises a respiration rate extraction unit for extracting the subject's respiration rate from said set of image data.

13. A system for obtaining vital sign information of a subject, said system comprising:
an imaging unit for detecting a set of image data from a skin portion of the subject, said image data allowing the extraction of the subject's heart rate,
a detection unit for detecting a set of detection data from a body portion of the subject, said detection data allowing the extraction of the subject's respiration rate or representing the subject's respiration rate, and
a device as claimed in claim 1 for obtaining vital sign information of the subject based on the detected set of image data and the detected set of detection data.

14. A method of obtaining vital sign information of a subject, said method comprising:
obtaining a set of image data detected from a skin portion of the subject allowing the extraction of the subject's heart rate and a set of detection data detected from a body portion of the subject allowing the extraction of the subject's respiration rate or representing the subject's respiration rate,
extracting the subject's heart rate from said set of image data by use of photoplethysmography,
predicting changes of the subject's heart rate based on the subject's respiration rate extracted from or represented by said set of detection data, and
controlling one or more settings of said heart rate extraction based on the predicted changes of the subject's heart rate.

15. A computer program comprising program code means for causing a computer to carry out the steps of the method as claimed in claim 14 when said computer program is carried out on a computer.
